(19) United States
(12) Patent Application Publication
(10) Pub. No.: US 2010/0210851 A1
(43) Pub. Date: Aug. 19, 2010

(54) PROCESS FOR PREPARING OXAZOLIDINE- AND OXAZOLIDINONE-AMINODIOLS

(76) Inventor: James C. Towson, Flemington, NJ (US)

Correspondence Address: Intervet/Schering-Plough Animal Health, Patent Dept. K-6-1, 1990, 2000 Galloping Hill Road, Kenilworth, NJ, 07033-0530, US

(21) Appl. No.: 12/669,645

(22) PCT Filed: Jul. 22, 2008
(86) PCT No.: PCT/US08/70696

§ 371 Date: Jan. 19, 2010

Related U.S. Application Data
(60) Provisional application No. 60/951,816, filed on Jul. 25, 2007.

(51) Int. Cl. C07C 231/12; C07D 263/04; C07D 263/06

(52) U.S. Cl. 548/230; 548/215; 564/212

(57) ABSTRACT

A method of preparing oxazolidine-protected and oxazolidinone-protected aminodiol compounds is disclosed. These compounds tend to be useful as intermediates in processes for making Florfenicol and related compounds.

### CROSS-REFERENCE TO RELATED PATENT APPLICATION

This patent claims priority to U.S. Provisional Patent Appl. No. 60/951,816 (filed Jul. 25, 2007). The entire text of that patent application is incorporated by reference into this patent.

### FIELD OF THE INVENTION

The present invention relates to a new process for preparing oxazolidine and oxazolidinone protected aminodiol compounds. These compounds are generally useful as intermediates in processes for making Florfenicol and related compounds.

### BACKGROUND OF THE INVENTION

Florfenicol is a broad spectrum antibiotic of Formula I

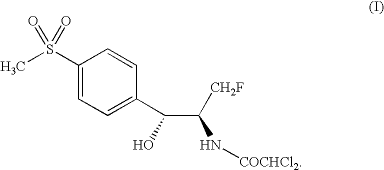

It has wide spread application in veterinary medicine for the treatment of both Gram positive and Gram negative bacteria as well as rickettsial infections. Florfenicol is also known as 2,2-Dichloro-N-[(1S,2R)-1-(fluoromethyl)-2-hydroxy-2-[4-(methylsulfonyl)phenyl]ethyl]-acetamide or [R—(R*,S*)]-2,2-dichloro-N-[1-(fluoromethyl)-2-hydroxy-2-[4-(methylsulfonyl)phenyl]ethyl]-acetamide.

U.S. Patent Published Application No. 2005/0075506 A1, the disclosure of which is incorporated herein by reference, describes the synthesis of Florfenicol intermediates of Formula II and their use in processes for making Florfenicol.

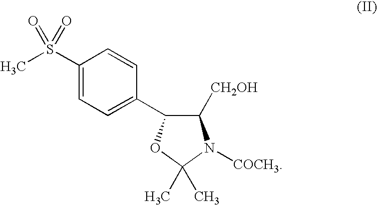

U.S. patent application Ser. Nos. 11/514,741, 11/515,278 and 11/515,135 also describe the preparation of Florfenicol intermediates of Formulas II (supra) and III:

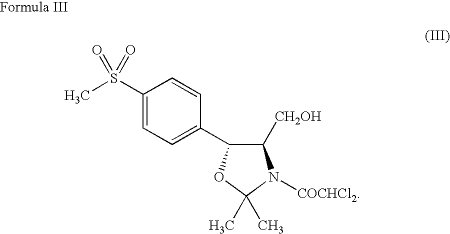

The primary advantage discussed therein is that the process eliminated the requirement in the prior art to use the expensive and difficult to isolate aminodiol sulfone (ADS) starting material. The ADS was generated in situ from a readily available and economical phenyl serine ester compound, then reacted further in the same reaction vessel to form the desired Florfenicol oxazolidine intermediates. Alternatively, as described in U.S. patent application Ser. No. 11/515,135, the use of or generation of the ADS was eliminated completely.

The present invention now discloses new processes to generate oxazolidine protected aminodiols and oxazolidinone protected aminodiols from compounds of Formula VII:

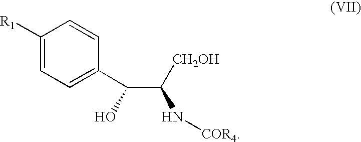

Applicants have now surprisingly found significant processing advantages for forming oxazolidine and oxazolidinone protected aminodiol compounds, allowing for more efficient and cost-saving processes. The present invention thus has the advantage of being an efficient and economical process for preparing Florfenicol, its analogs, and oxazolidine and oxazolidinone intermediates related thereto. The present invention is directed to oxazolidine and oxazolidinone protected aminodiol compounds and alternative methods of preparing useful intermediates included in the synthesis of Florfenicol.

### SUMMARY OF THE INVENTION

The present invention provides a process for preparing an oxazolidine protected aminodiol compound of Formula VI:

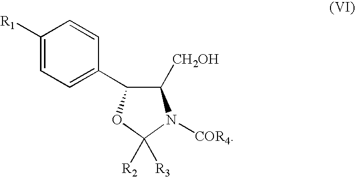

wherein:

R₁ is hydrogen, methylthio, methylsulfoxy, methylsulfonyl, fluoromethylthio, fluoromethylsulfoxy, fluoromethylsulfonyl, nitro, fluoro, bromo, chloro, acetyl, benzyl, phenyl, halo substituted phenyl, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, or a C₃₋₇ heterocyclic group;

R₂ is hydrogen, C₁₋₆ alkyl, C₁₋₆ haloalkyl, C₃₋₄ cycloalkyl, C₂₋₆ alkenyl, C₂₋₆ alkynyl, C₁₋₆ alkoxy, C₁₋₆ aralkyl, C₂₋₆ aralkenyl, aryl, or a C₃₋₇ heterocyclic group;

R₃ is hydrogen, C₁₋₆ alkyl, C₁₋₆ haloalkyl, C₃₋₄ cycloalkyl, C₂₋₆ alkenyl, C₂₋₆ alkynyl, C₁₋₆ alkoxy, C₁₋₆ aralkyl, C₂₋₆ aralkenyl, aryl, or a C₃₋₇ heterocyclic group; and

R₄ is hydrogen, C₁₋₆ alkyl, C₁₋₆ haloalkyl, C₁₋₆ dihaloalkyl, C₁₋₆ trihaloalkyl, CH₂Cl, CHCl₂, CCl₃, CH₂Br, CHBr₂, CBr₃, CH₂F, CHF₂, CF₃, C₃₋₈ cycloalkyl, C₃₋₄ cyclohaloalkyl, C₃₋₄ cyclodihaloalkyl, C₃₋₄ cyclotrihaloalkyl, C₂₋₆ alkenyl, C₂₋₆ alkynyl, C₁₋₆ alkoxy, C₁₋₆ aralkyl, C₂₋₆ aralkenyl, a C₃₋₇ heterocyclic group, benzyl, phenyl or phenyl alkyl wherein the phenyl or phenyl alkyl can be substituted by one or two halogens, C₁₋₆ alkyl, or C₁₋₆ alkoxy; or its pharmaceutically acceptable salt. In some embodiments, the process comprises the step of converting the compound of Formula VII to Florfenicol of Formula I.

In some embodiments, the process includes the steps of reacting a compound of Formula VII:

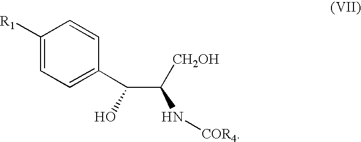

wherein R₁ and R₄ are as previously defined, in a vessel with an oxazolidine-forming solvent to form a reaction mixture, and adding an oxazolidine-forming reagent and an oxazolidine-promoting compound to the reaction mixture to form the oxazolidine protected aminodiol compound of Formula VI:

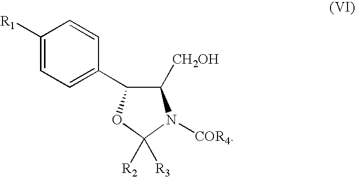

wherein R₁, R₂, R₃ and R₄ are as previously defined.

The present invention also provides a process for preparing an oxazolidinone protected aminodiol compound of Formula V:

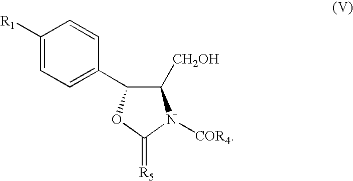

wherein R₁ and R₄ are as previously defined; and R₅ is oxygen, sulfur, or monosubstituted amino; or its pharmaceutically acceptable salt. In some embodiments, the process comprises the step of converting the compound of Formula V to Florfenicol of Formula I.

In some embodiments, the process includes the steps of reacting a compound of Formula VII:

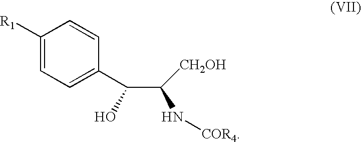

wherein R₁ and R₄ are as previously defined in a vessel with an oxazolidinone-forming solvent and adding an oxazolidinone-forming reagent and an oxazolidinone-promoting compound to form an oxazolidinone protected aminodiol compound of Formula V:

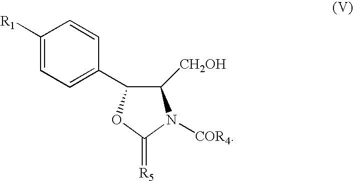

wherein R₁, R₄ and R₅ are as previously defined.

In some embodiments, a process of the present invention forms Florfenicol, related compounds, or both after the compounds of Formulas V and VI have been prepared.

The present invention also provides a compound of Formula:

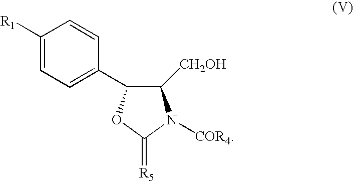

wherein R₁, R₄ and R₅ are as previously defined; or a pharmaceutically acceptable salt thereof.

In some embodiments, the compound of Formula V is the compound of Formula Vd:

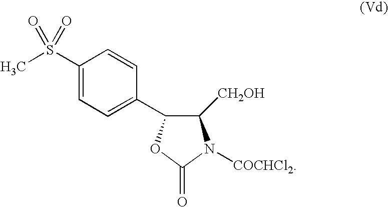

or a pharmaceutically acceptable salt thereof.

The present invention also provides a compound of Formula X:

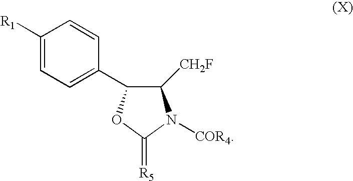

wherein R₁, R₄ and R₅ are as previously defined; or a pharmaceutically acceptable salt thereof.

In some embodiments, the compound of Formula V is the compound of Formula Xd:

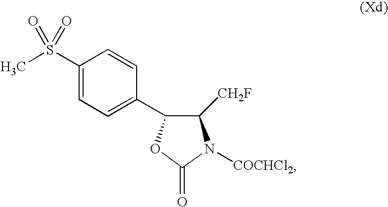

or a pharmaceutically acceptable salt thereof.

Further benefits of Applicants' invention will be apparent to one skilled in the art from reading this specification.

### DETAILED DESCRIPTION OF PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

When used herein and in the appended claims, the terms listed below, unless otherwise indicated, will be used and are intended to be defined as indicated immediately below. Definitions for other terms can occur throughout the specification. It is intended that all terms used include the plural, active tense and past tense forms of a term.

The term “acetyl” means a CH₃CO— radical.

The term “alcoholic solvent” includes C₁ to C₁₀ monoalcohols such as methanol, ethanol, and mixtures thereof, C₂ to C₁₀ dialcohols such as ethylene glycol and C₁ to C₁₀ trialcohols such as glycerin. Alternatively, the term alcoholic solvent includes such alcohol admixed with any suitable co-solvent (i.e., a second solvent added to the original solvent, generally in small concentrations, to form a mixture that has greatly enhanced solvent powers due to synergism). Such co-solvents can include other solvents which are miscible with the alcoholic solvent such as C₄ to C₁₀ alkanes, aromatic solvents such as benzene, toluene, and xylenes, halobenzenes such as chlorobenzene, and ethers such as diethylether, tert-butylmethylether, isopropylether and tetrahydrofuran, or mixtures of any of the above co-solvents.

The term “alkyl” means a saturated straight or branched alkyl such as methyl, ethyl, propyl, or sec-butyl. Alternatively, the number of carbons in an alkyl can be specified. For example, “C₁₋₆ alkyl” means an “alkyl” as described above containing 1, 2, 3, 4, 5 or 6 carbon atoms.

The term “C₂ alkenyl” means an unsaturated branched or unbranched hydrocarbon group having at least one double carbon-carbon (—C═C—) bond and containing 2, 3, 4, 5, or 6 carbon atoms. Example alkenyl groups include, without limitation, ethenyl, 1-propenyl, isopropenyl, 2-butenyl, 1,3-butadienyl, 3-pentenyl and 2-hexenyl, and the like.

The term “C₂₋₆ alkynyl” means an unsaturated branched or unbranched hydrocarbon group having at least one triple carbon-carbon (—C≡C—) bond and containing 2, 3, 4, 5, or 6 carbon atoms. Example alkynyl groups include, without limitation, ethynyl, 1-propynyl, 2-propynyl, 2-butynyl, 3-butynyl, 2-penten-4-ynyl, and the like.

The term “C₁₋₆ alkoxy” means an alkyl-O— group, where the term “alkyl” is defined herein. Example alkoxy groups include, without limitation, methoxy, ethoxy, propoxy (e.g., n-propoxy and isopropoxy), 1-butoxy, and the like,

The term “aryl” means phenyl, or phenyl substituted by C₁ to C₆ alkyl or “halo”, where phenyl and halo are as defined herein.

The term “C₁₋₆ aralkyl” means a C₁₋₆ alkyl as defined herein substituted by an aryl group that is any radical derived from an aromatic hydrocarbon by the removal of a hydrogen atom.

The term “C₂₋₆ aralkenyl” means a C₂₋₆ alkenyl as defined herein substituted by an aryl group that is any radical derived from an aromatic hydrocarbon by the removal of a hydrogen atom.

The term “bromo” means the chemical element bromine.

“Substituted benzyl” means benzyl substituted by C₁ to C₆ alkyl or “halo”, where benzyl is the univalent radical C₆H₅CH₂, formally derived from toluene (i.e., methylbenzene).

The term “chloro” means the chemical element chorine.

The term “C₃₋₈ cycloalkyl” means a saturated cyclic hydrocarbon group (i.e., a cyclized alkyl group) containing 3, 4, 5, 6, 7 or 8 carbon atoms. Example cycloalkyl groups include, without limitation, cyclopropyl, cyclobutyl, cyclopentyl, cyclohexyl and the like.

The term “C₃₋₈ cyclohaloalkyl” means a C₃₋₈ cycloalkyl as defined herein substituted by halo as defined herein.

The term “C₃₋₈ cyclodihaloalkyl” means a C₃₋₈ cycloalkyl as defined herein substituted twice by halo as defined herein where the halo atoms can be the same or different.

The term “C₃₋₈ cyclotrihaloalkyl” means a C₃₋₈ cycloalkyl as defined herein substituted thrice by halo as defined herein where the halo atoms can be the same or different.

The term “C₂ to C₁₀ dialcohol” means an alcohol containing two hydroxyl groups and 2, 3, 4, 5, 6, 7, 8, 9 or 10 carbon atoms.

The term “C₁₋₆ dihaloalkyl” means a C₁₋₆ alkyl as defined herein substituted twice by halo as defined herein where the halo atoms can be the same or different.

The term “fluoro” means the chemical element fluorine.

The term “fluoromethylsulfonyl” means a CH₂FSO₂— radical.

The term “fluoromethylsulfoxy” means a CH₂FSO— radical.

The term “fluoromethylthio” means a CH₂FS— radical.

The term “halo” or “halogen” means fluoro, chloro, bromo or iodo.

The term “haloalkyl” means an alkyl as described above wherein one or more hydrogens are replaced by halo as defined herein.

The term “halo substituted phenyl” means a phenyl as defined herein substituted by halo as defined herein.

The term “C₃₋₇ heterocyclic group” means a ring system radical where one or more of the ring-forming carbon atoms is replaced by a heteroatom, such as an oxygen, nitrogen, or sulfur atom, which include mono- or polycyclic (e.g., having 2 or more fused rings) ring systems as well as spiro ring systems. The ring system can contain 2, 3, 4, 5, or 6 carbon atoms and can be aromatic or non-aromatic.

The term “iodo” means the chemical element iodine.

The term “methylsulfonyl” means a CH₃SO₂— radical.

The term “methylsulfoxy” means a CH₃SO— radical.

The term “methylthio” means a CH₃S— radical.

The term “C₁ to C₁₀ monoalcohol” means an alcohol containing one hydroxyl group and 1, 2, 3, 4, 5, 6, 7, 8, 9 or 10 carbon atoms.

The term “monosubstituted amino” means an —NH₂ radical where one of its hydrogen is substituted by another atom or radical.

The term “nitro” means a —NO₂ radical.

The term “oxazolidine-promoting compound” means an acid or base that enhances, increases, accelerates or otherwise facilitates the reaction between the oxazolidine-forming reagent and the β-hydroxy amide compound.

The term “oxazolidine-forming reagent” means a reagent such that when reacted with a β-hydroxy amide compound forms an oxazolidine ring, where the oxygen of the β-hydroxy group and the nitrogen of the amide function are connected through a new carbon bond to form the oxaolidine ring.

The term “oxazolidine-forming solvent” means a solvent that by the nature of its dissolution properties enhances, increases, accelerates or otherwise facilitates the reaction between the oxazolidine-forming reagent and the β-hydroxy amide compound.

The term “oxazolidinone-promoting compound” means an acid or base that enhances, increases, accelerates or otherwise facilitates the reaction between the oxazolidinone-forming reagent and the β-hydroxy amide compound.

The term “oxazolidinone-forming reagent” means a reagent such that when reacted with a β-hydroxy amide compound forms an oxazolidinone ring where the oxygen of the β-hydroxy group and the nitrogen of the amide function are connected through a new carbon bond to form the oxaolidinone ring.

The term “oxazolidinone-promoting solvent” means a solvent that enhances, increases, accelerates of otherwise facilitates the reaction between the oxazolidinone-forming reagent and the β-hydroxy amide group to form an oxazolidinone ring.

The term “phenyl” means the monovalent radical C₆H₅— of benzene, which is the aromatic hydrocarbon C₆H₆.

The term “phenyl alkyl” means an alkyl as defined herein substituted by phenyl as defined herein.

The term “C₁ to C₁₀ trialcohol” means an alcohol containing three hydroxyl groups and 1, 2, 3, 4, 5, 6, 7, 8, 9 or 10 carbon atoms.

The term “C₁₋₆ trihaloalkyl” means a C₁₋₆ alkyl as defined herein substituted thrice by halo as defined herein where the halo atoms can be the same or different.

Throughout the specification and the appended claims, a given chemical formula or name shall encompass all stereo and optical isomers and racemates thereof, as well as mixtures in different proportions of the separate enantiomers, where such isomers and enantiomers exist, as well as pharmaceutically acceptable salts thereof and solvates thereof such as for instance hydrates. Isomers can be separated using conventional techniques, e.g. chromatography or fractional crystallization. The enantiomers can be isolated by separation of a racemic mixture, for example, by fractional crystallization, resolution or high-performance (or -pressure) liquid chromatography (HPLC). The diastereomers can be isolated by separation of isomer mixtures, for instance, by fractional crystallization, HPLC or flash chromatography. The stereoisomers also can be made by chiral synthesis from chiral starting materials under conditions which will not cause racemization or epimerization, or by derivatization, with a chiral reagent. The starting materials and conditions will be within the skill of one skilled in the art. All stereoisomers are included within the scope of the invention.

In one aspect, the present invention provides a process for preparing an oxazolidine protected aminodiol compound, or its pharmaceutically acceptable salt, of Formula VI:

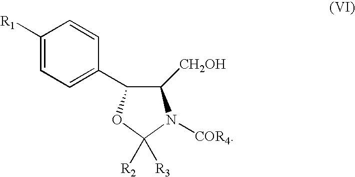

wherein:

R₁ is hydrogen, methylthio, methylsulfoxy, methylsulfonyl, fluoromethylthio, fluoromethylsulfoxy, fluoromethylsulfonyl, nitro, fluoro, bromo, chloro, acetyl, benzyl, phenyl, halo substituted phenyl, C₁₋₆ alkyl, C₁₋₆ haloalkyl, C₃₋₄ cycloalkyl, C₂₋₆ alkenyl, C₂₋₆ alkynyl, C₁₋₆ alkoxy, C₁₋₅ aralkyl, C₂₋₆ aralkenyl, or a C₃₋₇ heterocyclic group;

R₂ is hydrogen, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, aryl, or a C₃₋₇ heterocyclic group;

R₃ is hydrogen, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, aryl, or a C₃₋₇ heterocyclic group; and

R₄ is hydrogen, C₁₋₆ alkyl, C₁₋₆ haloalkyl, C₁₋₄ dihaloalkyl, C₁₋₆ trihaloalkyl, CH₂Cl, CHCl₂, CCl₃, CH₂Br, CHBr₂, CBr₃, CH₂F, CHF₂, CF₃, C₃₋₈ cycloalkyl, C₃₋₈ cyclohaloalkyl, C₃₋₈ cyclodihaloalkyl, C₃₋₄ cyclotrihaloalkyl, C₂₋₆ alkenyl, C₂₋₆ alkynyl, C₁₋₆ alkoxy, C₁₋₆ aralkyl, C₂₋₄ aralkenyl, a C₃₋₇ heterocyclic group, benzyl, phenyl or phenyl alkyl wherein the phenyl or phenyl alkyl can be substituted by one or two halogens, C₁₋₆ alkyl, or C₁₋₆ alkoxy; or a pharmaceutically acceptable salt thereof. In some embodiments, the process comprises the step of converting the compound of Formula VII to Florfenicol of Formula I.

In another aspect, the present invention provides a process for preparing an oxazolidinone protected aminodiol compound of Formula V:

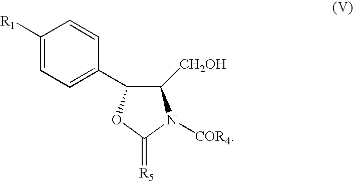

wherein R₁ and R₄ are as previously defined and R₅ is oxygen, sulfur or monosubstituted amino; or a pharmaceutically acceptable salt thereof. In some embodiments, the process comprises the step of converting the compound of Formula V to Florfenicol of Formula I.

The compounds of Formula V and VI are useful intermediates in the formation of Florfenicol and related compounds. The present invention thus has the advantage of being an efficient and economical process for preparing Florfenicol, its analogs, and oxazolidine or oxazolidinone intermediates related thereto.

In some embodiments of a process of the present invention, R₁ is methylthio, methylsulfoxy, or methylsulfonyl. In some such embodiments, R₁ is methylsulfonyl.

In some embodiments of a process of the present invention, R₂ and R₃ are hydrogen, methyl, ethyl or propyl. In some such embodiments, R₂ and R₃ are methyl.

In some embodiments of a process of the present invention, R₄ is CH₂Cl, CHCl₂, CCl₃, CH₂Br, CHBr₂, CBr₃, CH₂F, CHF₂, or CF₃. In some such embodiments, R₄ is CH₂Cl, CHCl₂, or CCl₃. In some such embodiments, R₄ is CHCl₂.

In some embodiments of a process of the present invention, R₅ is oxygen.

In some embodiments, the process for preparing an oxazolidine protected aminodiol of Formula VI includes the steps of reacting a compound of Formula VII:

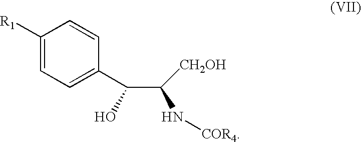

wherein R₁ and R₄ are as previously defined, in a vessel with an oxazolidine-forming solvent to form a reaction mixture, and adding an oxazolidine-forming reagent and oxazolidine-promoting compound to the reaction mixture to form the oxazolidine aminodiol protected compound of Formula VI.

In some such embodiments, the compounds of Formulas VIIa and VIIb are starting materials:

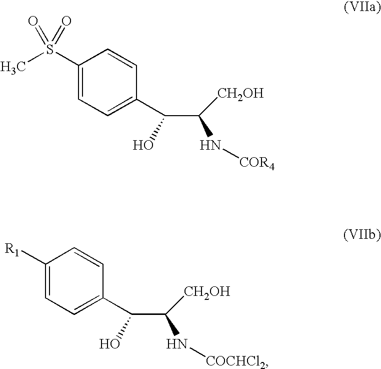

wherein R₁ and R₄ are as previously defined.

In some embodiments, the starting material is the commercially available, economical and widely known antibiotic thiamphenicol of Formula IV:

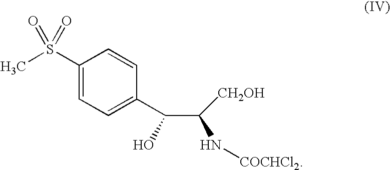

In some embodiments, the compound of Formula VII reacts in an oxazolidine-forming solvent, such as and without limitation, acetone, methylene chloride, ethyl acetate, tetrahydrofuran, ether, toluene, xylene, hexane and a mixture thereof. In some such embodiments, the oxazolidine-forming solvent comprises toluene. An oxazolidine-forming reagent, such as and without limitation, formaldehyde, acetone, 2-methoxypropene, 2,2-dimethoxypropane, 2,2-diethoxypropane and a mixture thereof, is then added. In some embodiments, the oxazolidine-forming reagent comprises acetone.

In some embodiments, the oxazolidine-forming solvent comprises toluene and the oxazolidine-forming reagent comprises acetone. In some such embodiments, toluene and acetone are present in a ratio of from about 0.5:1 to about 3:1. In some such embodiments, the ratio is about 1:1.

The presence of an acid or a base, designated herein as an oxazolidine-promoting compound, such as and without limitation, potassium carbonate, sodium carbonate, trimethylamine, triethylamine, p-toluene sulfonic acid, methanesulfonic acid, acetic acid, hydrochloric acid, sulfuric acid, nitric acid, phosphoric acid, and a mixture thereof facilitates the reaction with the oxazolidine-forming reagent. In some embodiments, the oxazolidine-promoting compound is potassium carbonate, triethylamine, p-toluene sulfonic acid or a mixture thereof. In some embodiments, the oxazolidine-promoting compound is potassium carbonate, triethylamine, or a mixture thereof.

In some embodiments, the oxazolidine-forming reaction is carried out at a temperature from about 40° C. to about 110° C. In some embodiments, the temperature is from about 65° C. to about 85° C.

In some embodiments, the compound of Formula VI corresponds to the compound of Formula VIa:

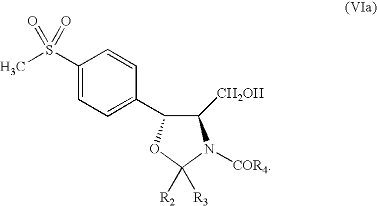

wherein R₂, R₃ and R₄ are as previously defined.

In some embodiments, the compound of Formula VI corresponds to the compound of Formula VIb:

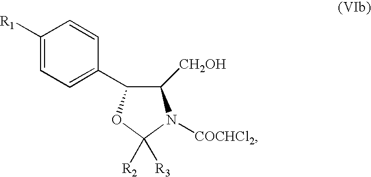

wherein R₁, R₂ and R₃ are as previously defined.

In some embodiments, Florfenicol is the desired end product. In some such embodiments, the compound of Formula VI corresponds to the compound of Formula VIc:

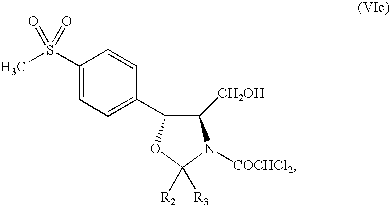

wherein R₂ and R₃ are as previously defined. In still further such embodiments, the compound of Formula VI corresponds to the compound of Formula III.

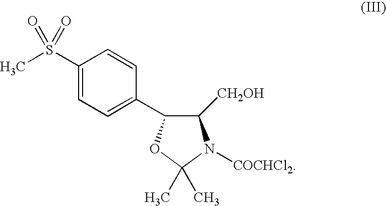

Once the compound of Formula VI has been prepared, one can use this compound as an intermediate for preparing Florfenicol and related compounds. Therefore, in continuing the process to prepare Florfenicol and related compounds, the process involves fluorinating the compound of Formula VI with a fluorinating agent, with or without isolation (i.e. in situ), in the presence of an organic solvent to obtain the compound of Formula VIII:

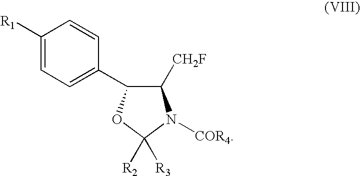

wherein R₁, R₂, R₃ and R₄ are as previously defined.

In some such embodiments, suitable fluorinating agents include, without limitation, sodium fluoride, potassium fluoride, cesium fluoride, tetrabutylammonium fluoride, 1,1,2,2,3,3,4,4,4-nonafluoro-1-butanesulfonyl fluoride, chloromethyl-4-fluoro-1,4-diazoniabicyclo[2.2.2]octane bis-(tetrafluoroborate), N-(2-chloro-1,1,2-trifluoroethyl)diethylamine, N-(2-chloro-1,1,2-trifluoroethyDdimethylamine, N-(2-chloro-1,1,2-trifluoroethyl)dipropylamine, N-(2-chloro-1,1,2-trifluoroethyl)pyrrolidine, N-(2-chloro-1,1,2-trifluoroethyl)-2-methylpyrrolidine, N-(2-chloro-1,1,2-trifluoroethyl)-4-methylpiperazine, N-(2-chloro-1,1,2-trifluoroethyl)-morpholine, N-(2-chloro-1,1,2-trifluoroethyl)piperidine, 1,1,2,2-tetrafluoroethyl-N,N-dimethylamine, (diethylamino) sulfur trifluoride, Bis-(2-methoxyethyl)aminosulfur trifluoride, N,N-diethyl-1,1,2,3,3,3-hexafluoro-1-propanamine (commonly referred to as Ishikawa Reagent) and a mixture thereof. In some embodiments, the fluorinating agent comprises N,N-diethyl-1,1,2,3,3,3-hexafluoro-1-propanamine.

In some embodiments, the fluorinated compound of Formula VIII corresponds to the compound of Formula VIIIa:

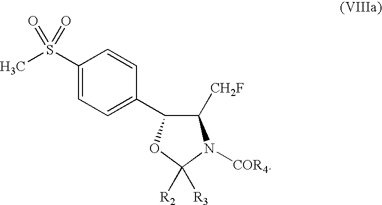

wherein R₂, R₃ and R₄ are as previously defined.

In some embodiments, the fluorinated compound of Formula VIII corresponds to the compound of Formula VIIIb:

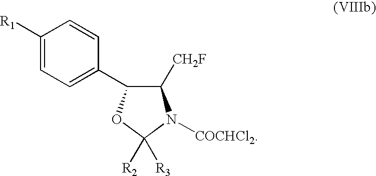

wherein R₁, R₂ and R₃ are as previously defined.

In some embodiments, when Florfenicol is the desired end product, the fluorinated compound of Formula VIII corresponds to the compound of Formula VIIIc:

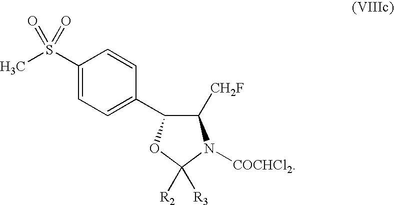

wherein R₂ and R₃ are as previously defined.

In some embodiments, when Florfenicol is the desired end product, the fluorinated compound of Formula VIII corresponds to the compound of Formula VIIId:

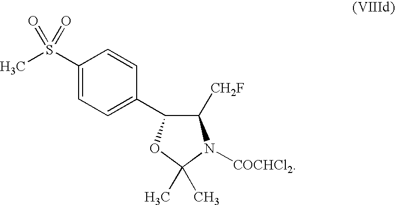

Once the compound of Formula VIII has been prepared, one can use this compound as an intermediate for preparing Florfenicol and related compounds. Therefore, in continuing the process to prepare Florfenicol and related compounds, the process then involves hydrolyzing, with or without isolation (i.e. in situ), with an acid catalyst or a basic catalyst to form the compound of Formula IX:

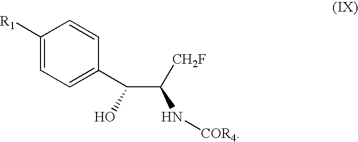

wherein R₁ and R₄ are as previously defined.

In some embodiments, hydrolysis is selective, i.e., hydrolysis of a compound at a specific location of the compound, where hydrolysis refers to the addition of water to the compound, thereby causing the splitting of the compound.

A wide range of acid catalysts can be employed in carrying out the process of the present invention. A non-limiting list of suitable acid catalysts include inorganic acids, such as dilute aqueous hydrochloric acid, sulfuric acid, nitric acid, phosphoric acid, and a mixture thereof, as well as organic acids, such as acetic acid, methanesulfonic acid, p-toluene sulfonic acid and a mixture thereof.

Similarly, a wide range of basic catalysts can be employed in carrying out the process of the present invention. A non-limiting list of suitable basic catalysts include inorganic bases, such as LiOH, NaOH, KOH, Li₂CO₃, Na₂CO₃, K₂CO₃ and a mixture thereof, as well as organic bases, such as sodium methoxide, sodium ethoxide, potassium methoxide, potassium ethoxide and a mixture thereof.

In some embodiments, the hydrolyzing step is carried out with the compound of Formula VIII and the acid catalyst or the basic catalyst in an organic solvent, water or a mixture of an organic solvent and water. A non-limiting list of organic solvents useful in the hydrolyzing step include acetone, methanol, ethanol, propanol, isopropanol, methylene chloride, ethyl acetate, tetrahydrofuran and mixtures thereof.

In some embodiments, the compound of Formula IX formed by the hydrolyzing step corresponds to the compound of Formula IXa:

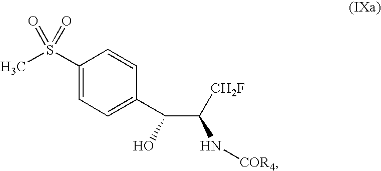

wherein R₄ is as previously defined.

In some embodiments, the compound of Formula IX formed by the hydrolyzing step corresponds to the compound of Formula IXb:

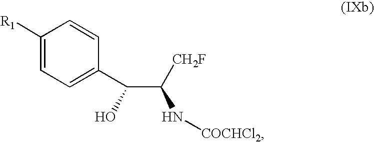

wherein R₁ is as previously defined.

In some embodiments, when Florfenicol is the desired end product, the compound of Formula IX formed by the hydrolyzing step corresponds to Florfenicol of Formula I:

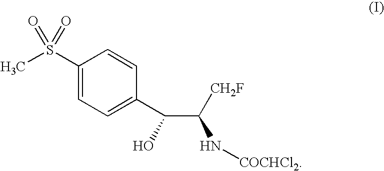

After the compound of Formula IX has been prepared the compound of Formula IX optionally can be purified. In some embodiments, purifying the compound of Formula IX involves using a mixture of a C₁₋₁₀ alkyl monoalcohol, a C₁₋₁₀ alkyl dialcohol or a C₁₋₁₀ alkyl trialcohol and water to form the purified compound of Formula IX. A non-limiting list of C₁₋₁₀ monoalcohols includes methanol, ethanol, propanol, isopropanol, butanol, sec-butanol, t-butanol, pentanol and a mixture thereof. A non-limiting list of C₁₋₁₀ dialcohols includes ethylene glycol, propylene glycol, butylene glycol and a mixture thereof. A non-limiting example of a C₁₋₁₀ trialcohol is glycerin.

In some embodiments, the process for preparing an oxazolidinone protected aminodiol compound of Formula V includes the steps of reacting a compound of Formula VII:

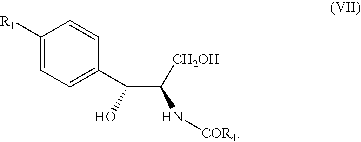

wherein R₁ and R₄ are as previously defined, in a vessel with an oxazolidinone-forming solvent to form a reaction mixture, and adding an oxazolidinone-forming reagent and an oxazolidinone-promoting compound to the reaction mixture to form the oxazolidinone protected aminodiol of Formula V:

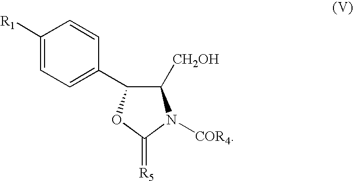

wherein R₁, R₄ and R₅ are as previously defined.

In some embodiments, the oxazolidinone-forming solvent comprises, for example and without limitation, ethyl acetate, acetone, tetrahydrofuran, ether, methylene chloride, methanol, ethanol, propanol, isopropanol, toluene, xylene, hexane or a mixture thereof. In some embodiments, the oxazolidinone-forming solvent comprises methanol.

In some embodiments, the oxazolidinone-forming reagent comprises phosgene, triphosgene, trichloromethyl chloroformate, urea, thiourea, p-nitrophenyl chloroformate, methyl chloroformate, ethyl chloroformate, propyl chloroformate, N, N-carbonyldiimidazole, dimethyl carbonate, diethyl carbonate, dipropyl carbonate, dibutyl carbonate or a mixture thereof. In some embodiments, the oxazolidinone-forming reagent comprises dimethyl carbonate, diethyl carbonate, dipropyl carbonate, dibutyl carbonate or a mixture thereof. In some embodiments, the oxazolidinone-forming reagent comprises dimethyl carbonate, diethyl carbonate, or a mixture thereof.

In some embodiments, the oxazolidinone-forming reagent and the compound of Formula VII have a molar ratio of from about 0.5:1 to about 3:1. In some embodiments, the molar ratio is about 1:1.

The presence of an acid or a base, designated herein as an oxazolidinone-promoting compound, such as, and without limitation, potassium carbonate, sodium carbonate, sodium methoxide, sodium ethoxide, trimethylamine, triethylamine, p-toluene sulfonic acid, methanesulfonic acid, acetic acid, hydrochloric acid, sulfuric acid, nitric acid, phosphoric acid, or a mixture thereof, facilitates the reaction with the oxazolidinone-forming reagent. In some embodiments, the oxazolidinone-promoting compound comprises potassium carbonate, triethylamine or a mixture thereof.

In some embodiments, the compound of Formula V corresponds to the compound of Formula Va:

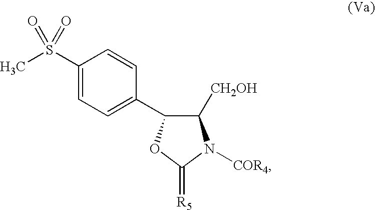

wherein R₄ and R₅ are as previously defined.

In some embodiments, the compound of Formula V corresponds to the compound of Formula Vb:

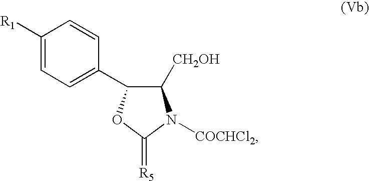

wherein R₁ and R₅ are as previously defined.

In some embodiments, when Florfenicol is the desired end product, the compound of Formula V corresponds to the compound of Formula Vc:

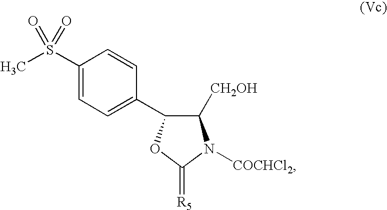

wherein R₅ is as previously defined.

In some embodiments, when Florfenicol is the desired end product, the compound of Formula V corresponds to the compound of Formula Vd:

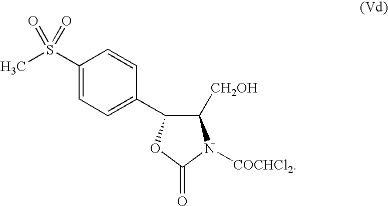

After the compound of Formula V has been prepared, one can use this compound as an intermediate for preparing Florfenicol and related compounds. Thus, in continuing the process to prepare Florfenicol and related compounds, the process involves reacting the oxazolidinone protected aminodiol of Formula V, with or without isolation (i.e., in situ), with a fluorinating agent to form a compound of Formula X:

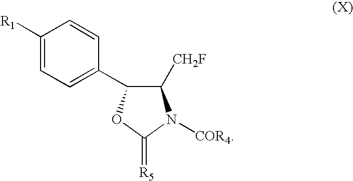

wherein R₁, R₄ and R₅ are as previously defined.

Suitable fluorinating agents and organic solvents useful during this part of the process are, for example and without limitation, those previously described above.

In some embodiments, the compound of Formula X corresponds to the compound of Formula Xa:

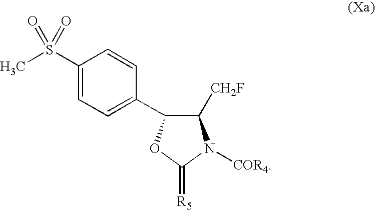

wherein R₄ and R₅ are as previously defined.

In some embodiments, the compound of Formula X corresponds to the compound of Formula Xb:

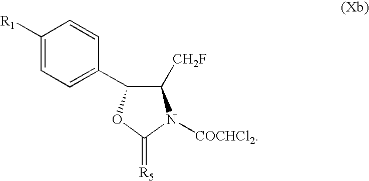

wherein R₁ and R₅ are as previously defined.

In some embodiments, when Florfenicol is the desired end product, the compound of Formula X corresponds to the compound of Formula Xc:

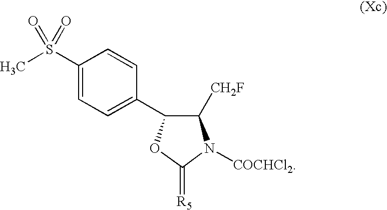

wherein R₅ is as previously defined.

In some embodiments, when Florfenicol is the desired end product, the compound of Formula X corresponds to the compound of Formula Xd:

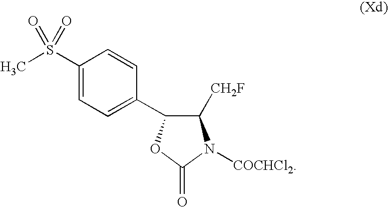

After the compound of Formula X has been prepared, it is hydrolyzed, with or without isolation (i.e. in situ), with an acid catalyst or a basic catalyst to form the compound of Formula IX:

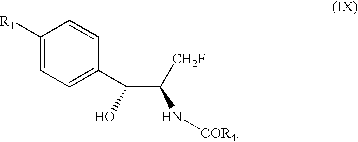

wherein R₁ and R₄ are as previously defined.

A wide range of acids can be employed in carrying out the process of the invention, such as and without limitation, those previously described above. Similarly, a wide range of bases can be employed in carrying out the process of the invention, such as and without limitation, those previously described above.

In some embodiments, the hydrolyzing step is carried out with the compound of Formula X and the acid catalyst or the basic catalyst in an organic solvent, water or a mixture of an organic solvent and water. A non-limiting list of organic solvents are, for example and without limitation, those previously described above.

In some embodiments, the compound of Formula IX corresponds to the compound of Formula IXa:

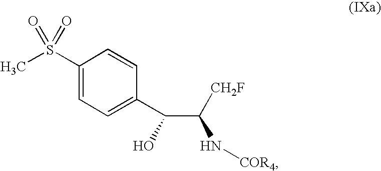

wherein R₄ is as previously defined.

In some embodiments, the compound of Formula IX corresponds to the compound of Formula IXb:

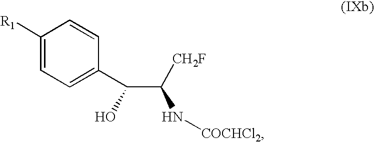

wherein R₁ is as previously defined.

In some embodiments, when Florfenicol is the desired end product, the compound of Formula IX corresponds to Florfenicol of Formula I:

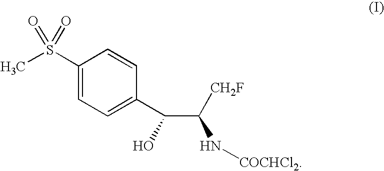

After the compound of Formula IX is made and if necessary, it can optionally be purified by the process as described herein. When Florfenicol is the desired end product, the purified compound corresponding to Formula IX is the compound of Formula I.

In some embodiments of a process of the present invention, the oxazolidine protected aminodiol compound of Formula VI or the oxazolidinone protected aminodiol compound of Formula V is substantially formed (i.e., the reaction is greater than 95% completed) over from about 2 to about 18 hours.

In some embodiments of a process of the present invention, the fluorinating agent such as N,N-diethyl-1,1,2,3,3,3-hexafluoro-1-propanamine and the compound according to Formula VI have a molar ratio of from about 1:1 to about 2:1. In some embodiments, the molar ratio of the N,N-diethyl-1,1,2,3,3,3-hexafluoro-1-propanamine to the compound of Formula VI is about 1.5:1.

In some embodiments of a process of the present invention, the fluorinating agent such as N,N-diethyl-1,1,2,3,3,3-hexafluoro-1-propanamine and the compound according to Formula V have a molar ratio of from about 1:1 to about 2:1. In some embodiments, the molar ratio of the N,N-diethyl-1,1,2,3,3,3-hexafluoro-1-propanamine to the compound of Formula V is about 1.5:1.

In some embodiments of a process of the present invention, suitable organic solvents for the fluorinating step include, without limitation, 1,2-dichloroethane, methylene chloride, chloroform, chlorobenzene, chlorinated hydrocarbons and mixtures thereof. In some embodiments, the organic solvent comprises methylene chloride.

In some embodiments of a process of the present invention, the fluorinating step is carried out at a temperature of from about 80° C. to about 110° C., and at a pressure of about 60 psi.

In some embodiments of a process of the present invention, the acid catalyst of the hydrolyzing step comprises an inorganic acid, an organic acid or a mixture thereof. In some embodiments of a process of the present invention, the acid catalyst comprises p-toluene sulfonic acid. In some embodiments of a process of the present invention, the acid catalyst comprises methanesulfonic acid.

In some embodiments of a process of the present invention, the acid catalyst for the hydrolyzing step comprises an inorganic base, an organic base or a mixture thereof. In some embodiments, the basic catalyst comprises K₂CO₃. In some embodiments, the basic catalyst comprises LiOH.

In some embodiments of a process of the present invention, the organic solvent for the hydrolyzing step comprises tetrahydrofuran. In some embodiments of a process of the present invention, the organic solvent comprises methylene chloride. In some embodiments of a process of the present invention, the solvent is the mixture of the organic solvent and water. In some such embodiments, the organic solvent is methylene chloride.

The hydrolysis step of a process of the present invention can be carried out at a temperature up to about 100° C. That is to say, hydrolysis is performed at a temperature less than or equal to about 100° C. In some embodiments, the temperature is less than about 80° C.

In some embodiments of a process of the present invention, the hydrolyzing step further comprises heating the compound of Formula VIII or Formula X with the acid catalyst or the basic catalyst in a mixture of an organic solvent and water at a temperature less than about 100° C.

Other suitable hydrolyzing steps will be apparent to those of ordinary skill in the art.

In some embodiments of a process of the present invention, the resultant compound of the fluorinating step (e.g., the compound of Formula VI or Formula X), the resultant compound of the hydrolyzing step (e.g., the compound of Formula VII or Formula IX), or any combination thereof, is isolated. In some embodiments, the resultant compound or any combination thereof is not isolated (i.e., is generated in situ).

In some embodiments of a process of the present invention, the C₁₋₁₀ monoalcohol for the purifying step comprises isopropanol. In some embodiments of a process of the present invention, the C₁₋₁₀ dialcohol of the purifying step comprises propylene glycol. In some embodiments of a process of the present invention, the C₁₋₁₀ trialcohol of the purifying step comprises glycerin.

In some embodiments of a process of the present invention, the purifying step comprises using a mixture of alcohol and water. In some embodiments, the mixture comprises methanol, ethanol, propanol, isopropanol, butanol, sec-butanol, t-butanol, pentanol, ethylene glycol, propylene glycol, butylene glycol, glycerin or a mixture thereof. In some embodiments, the alcohol, such as isopropanol, and water are present in a ratio of from about 1:5 to about 5:1. In some embodiments, the ratio of alcohol to water is about 1:1. In some embodiments, the alcohol comprises isopropanol and the ratio of the isopropanol to water mixture is about 1:1. In some embodiments, the compound of Formula IX and the about 1:1 isopropanol and water mixture have a weight to volume ratio of from about 1:1 to about 10:1. In some embodiments, the weight to volume ratio of the compound of Formula IX to the about 1:1 isopropanol and water mixture is about 1:4.6.

In some embodiments of the purifying step of a process of the present invention, the compound of Formula IX is dissolved in an about 1:1 isopropanol and water mixture, and the purifying step has a dissolution temperature that is the reflux point of the 1:1 isopropanol and water mixture. In some embodiments, the compound of Formula IX is dissolved in an about 1:1 isopropanol and water mixture, where the compound of Formula IX and the about 1:1 isopropanol and water mixture have a weight to volume ratio of about 1:4.6, and heated to the reflux point of the mixture. The resultant solution is clarified by filtration with active carbon and a filter, then cooled at a temperature of from about 10° C. to about 30° C. to obtain crystallized compound of Formula IX that is pure. As used herein, the terms “pure” or “purified” means reduced levels of impurities and improved color compared to unpurified compound. In some embodiments, the solution is cooled to a temperature of from about 20° C. to about 25° C. to crystallize the purified compound of Formula IX from the solution. In some embodiments, the purified compound of Formula IX crystallized from the solution is Florfenicol.

In another aspect, the present invention provides a compound of Formula V, having a structure of:

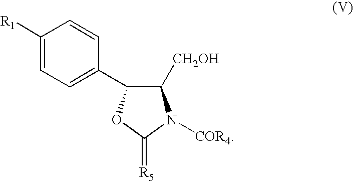

wherein R₁, R₄, and R₅ are as previously defined; or a pharmaceutically acceptable salt thereof. In some embodiments, the compound of Formula V is the compound of Formula Vd:

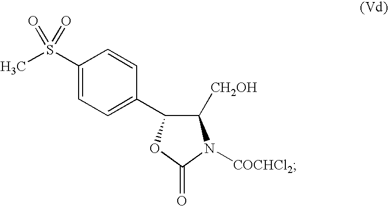

or
a pharmaceutically acceptable salt thereof

In another aspect, the present invention provides a compound of Formula X having a structure of:

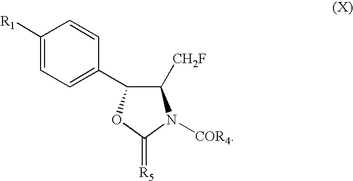

wherein R₁, R₄ and R₅ are as previously defined, with the proviso that if R₄ is O-t-butyl and R₅ is O, then R₁ is not Br, CH₃SO₂ or CH₃S; or a pharmaceutically acceptable salt thereof. In some embodiments, the compound of Formula X is the compound of Formula Xd:

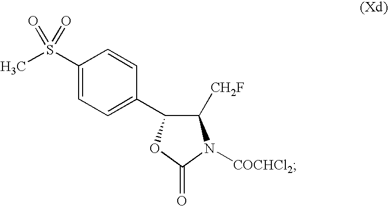

or a pharmaceutically acceptable salt thereof.

### EXAMPLES

The following hypothetical preparative examples are representative examples of a process and compounds of the present invention. While the present invention has been described with specificity in accordance with certain embodiments of the present invention, the following examples further serve only to exemplify and illustrate the present invention and are not intended to limit or restrict the effective scope of the present invention.

The term “C₁₋₆ trihaloalkyl” means a C₁₋₆ alkyl as defined herein substituted thrice by halo as defined herein where the halo atoms can be the same or different.

### Example 1

Preparation of 3-(dichloroacetyl)-4(R)-(hydroxymethyl)-2,2-dimethyl-5(R)-[4-(methylsulfonyl)phenyl]oxazolidine (Compound III). Thiamphenicol (Compound IV) (about 10 g, 0.0281 moles) and triethylamine can be reacted in toluene (about 50 mL) and acetone (about 50 mL) at a temperature of from about 70° C. to about 80° C. for about 16 hours to provide a reaction mixture. Following cooling to a temperature of from about 20° C. to about 25° C., evaporation of the solvent, washing with toluene and water then drying, the reaction mixture can yield 3-(dichloroacetyl)-4(R)-(hydroxymethyl)-2,2-dimethyl-5(R)-[4-(methylsulfonyl)phenyl]oxazolidine (Compound III).

### Example 2

Preparation of 3-(dichloroacetyl)-4(R)-(hydroxymethyl)-2,2-dimethyl-5(R)-[4-(methylsulfonyl)phenyl]oxazolidine (Compound III). Thiamphenicol (Compound IV) (about 5 g, 0.0140 moles), 2,2-dimethoxypropane (about 2.2 g, 0.0211 moles) and p-toluene sulfonic acid can be reacted in toluene (about 50 mL) at a temperature of from about 75 to about 85° C. over about 18 hours to provide a reaction mixture. Following cooling to a temperature of from about 20° C. to about 25° C., evaporation of the solvent, washing with toluene and water then drying, the reaction mixture can yield 3-(dichloroacetyl)-4(R)-(hydroxymethyl)-2,2-dimethyl-5(R)-[4-(methylsulfonyl)phenyl]oxazolidine (Compound III).

### Example 3

Preparation of 3-(dichloroacetyl)-4(S)-(fluoromethyl)-2,2-dimethyl-5(R)-[4-(methylsulfonyl)phenyl]oxazolidine (Compound VIIId). 3-(Dichloroacetyl)-4(R)-(hydroxymethyl)-2,2-dimethyl-5(R)-[4-(methylsulfonyl)phenyl]oxazolidine (Compound III) (about 10 g, 0.0252 moles) in methylene chloride (about 70 ml) can be reacted with N,N-diethyl-1,1,2,3,3,3-hexafluoro-1-propanamine (Ishikawa Reagent) (about 10.7 g, 0.0478 moles) at a temperature of from about 95° C. to about 105° C. over about 4 hours to provide a reaction mixture. Following cooling to a temperature of from about 20° C. to about 25° C., addition to sodium hydroxide (about 1.5 g) in water (about 330 mL), separation of the methylene chloride layer, distillation and replacement of methylene chloride by isopropanol (about 50 mL), then concentration of the isopropanol, the reaction mixture can precipitate the desired product. Following filtration, washing with water and isopropanol, then drying, the desired product can yield 3-(dichloroacetyl)-4(S)-(fluoromethyl)-2,2-dimethyl-5(R)-[4-(methylsulfonyl)phenyl]oxazolidine (Compound VIIId).

### Example 4

Preparation of 3-(dichloroacetyl)-4(S)-(fluoromethyl)-2,2-dimethyl-5(R)-[4-(methylsulfonyl)phenyl]oxazolidine (Compound VIIId). Thiamphenicol (Compound IV) (about 10 g, 0.0281 moles), acetone (about 10 mL) and p-toluene sulfonic acid in methylene chloride (about 200 mL) can be reacted over about 18 hours at reflux to form 3-(dichloroacetyl)-4(R)-(hydroxymethyl)-2,2-dimethyl-5(R)-[4-(methylsulfonyl)phenyl]oxazolidine (Compound III) in solution. Addition of anhydrous sodium sulfate and charcoal to Compound III in solution, followed by filtration, and concentration of the solution to about 100 mL can yield a dry solution of Compound III, which then can be reacted with N,N-diethyl-1,1,2,3,3,3-hexafluoro-1-propanamine (Ishikawa Reagent) (about 9.4 g, 0.0421 moles) at a temperature of from about 95° C. to about 105° C. for over about 4 hours to provide a reaction mixture. Following cooling to a temperature of from about 20° C. to about 25° C., addition to sodium hydroxide (about 1 g) in water (about 330 mL), separation of the methylene chloride layer, evaporation of the methylene chloride, washing with water and isopropanol then drying, the reaction mixture can yield 3-(dichloroacetyl)-4(S)-(fluoromethyl)-2,2-dimethyl-5(R)-[4-(methylsulfonyl)phenyl]oxazolidine (Compound VIIId).

### Example 5

Preparation of Florfenicol (Compound I). 3-(Dichloroacetyl)-4(S)-(fluoromethyl)-2,2-dimethyl-5(R)-[4-(methylsulfonyl)phenyl]oxazolidine (about 10 g, 0.0251 moles) can be hydrolyzed in methylene chloride (about 50 mL) and water (about 20 mL) containing p-toluene sulfonic acid at about 60° C. over several (e.g., 4 to 8) hours to provide a reaction mixture. Following removal of the methylene chloride by distillation and cooling to a temperature of from about 20° C. to about 25° C., the reaction mixture can precipitate the product. Following filtration, washing with water (about 20 mL) and toluene (about 20 mL) then drying, the product can yield Florfenicol (Compound I).

### Example 6

Purification of Florfenicol (Compound I). Florfenicol (Compound I) (about 25 g, 0.0700 moles) can be dissolved in water (about 60 mL) and isopropanol (about 60 mL) at reflux to provide a mixture. Following addition of charcoal, clarification by filtration, cooling to a temperature of from about 20° C. to about 25° C., filtration of the solids, washing with about 1:1 water/isopropanol (about 20 mL) then drying, the mixture can yield pure Florfenicol (Compound D.

### Example 7

Preparation of Florfenicol (Compound I). 3-(Dichloroacetyl)-4(R)-(hydroxymethyl)-2,2-dimethyl-5(R)-[4-(methylsulfonyl)phenyl]oxazolidine (Compound III) (5 g, 0.0126 moles) in methylene chloride (about 50 ml) can be reacted with N,N-diethyl-1,1,2,3,3,3-hexafluoro-1-propanamine (Ishikawa Reagent) (about 4.2 g, 0.0188 moles) at a temperature of from about 95° C. to about 105° C. over about 4 hours a reaction mixture. Following cooling to a temperature of from about 20° C. to about 25° C., quenching with about 25% aqueous sodium hydroxide and water (about 75 mL) then separation of the methylene chloride layer, the reaction mixture gives a solution of 3-(dichloroacetyl)-4(S)-(fluoromethyl)-2,2-dimethyl-5(R)-[4-(methylsulfonyl)phenyl]oxazolidine (Compound VIIId). Following addition of water and potassium carbonate with heating to a temperature of from about 50° C. to about 60° C. for about 10 hours, cooling to a temperature of from about 20° C. to about 25° C., filtration of the solids, washing with water and toluene then drying, the solution of Compound VIIId can yield Florfenicol (Compound I).

### Example 8

Preparation of 3-(dichloroacetyl)-4(R)-(hydroxymethyl)-5(R)-[4-(methylsulfonyl)phenyl]-2-oxazolidinone (Compound Vd). Thiamphenicol (Compound IV) (about 10 g, 0.0281 moles) can be reacted with diethylcarbonate (about 3.7 g, 0.0313 moles) and potassium carbonate in methanol (about 100 mL) at reflux over about 6 hours to provide a reaction mixture. Following cooling to a temperature of from about 20° C. to about 25° C., evaporation of the solvent, washing with toluene and water then drying, the reaction mixture can yield 3-(dichloroacetyl)-4(R)-(hydroxymethyl)-5(R)-[4-(methylsulfonyl)phenyl]-2-oxazolidinone (Compound (Vd)).

### Example 9

Preparation of 3-(dichloroacetyl)-4(R)-(hydroxymethyl)-5(R)-[4-(methylsulfonyl)phenyl]-2-oxazolidinone (Compound Vd). Thiamphenicol (Compound IV) (about 5 g, 0.0140 moles) can be reacted with ethyl chloroformate (about 2.6 g, 0.0185 moles) and triethylamine in methanol (about 50 mL) at a temperature of from about 0° C. to about 10° C. over about 10 hours to provide a reaction mixture. Following addition of water and concentration of the solvent, the reaction mixture can precipitate the product. Following washing with toluene and water then drying, the product can yield 3-(dichloroacetyl)-4(R)-(hydroxymethyl)-5(R)-[4-(methylsulfonyl)phenyl]-2-oxazolidinone (Compound Vd).

### Example 10

Preparation of 3-(dichloroacetyl)-4(S)-(fluoromethyl)-5(R)-[4-(methylsulfonyl)phenyl]-2-oxazolidinone (Compound Xd). 3-(Dichloroacetyl)-4(R)-(hydroxymethyl)-5(R)-[4-(methylsulfonyl)phenyl]-2-oxazolidinone (Compound Vd) (about 10 g, 0.0260 moles) in methylene chloride (about 100 ml) can be reacted with N,N-diethyl-1,1,2,3,3,3-hexafluoro-1-propanamine (Ishikawa Reagent) (about 8.7 g, 0.039 moles) at a temperature of from about 95° C. to about 105° C. over about 4 hours to provide a reaction mixture. Following cooling to a temperature of from about 20° C. to about 25° C., addition to sodium hydroxide (about 1.2 g) in water (about 300 mL), separation of the methylene chloride layer, distillation and replacement of methylene chloride by isopropanol and addition of water, the reaction mixture can precipitate the desired product. Following filtration, washing with water and isopropanol then drying, the product can yield 3-(dichloroacetyl)-4(S)-(fluoromethyl)-5(R)-[4-(methylsulfonyl)phenyl]-2-oxazolidinone (Compound Xd).

### Example 11

Preparation of 3-(dichloroacetyl)-4(S)-(fluoromethyl)-5(R)-[4-(methylsulfonyl)phenyl]-2-oxazolidinone (Compound Xd). Thiamphenicol (Compound IV) (about 5 g, 0.0140 moles) can be reacted with ethyl chloroformate (about 2.6 g, 0.0185 moles) and triethylamine in methylene chloride (about 250 mL) at ambient room temperature over several hours to yield a solution of 3-(dichloroacetyl)-4(R)-(hydroxymethyl)-5(R)-[4-(methylsulfonyl)phenyl]-2-oxazolidinone (Compound Vd). Following drying over anhydrous sodium sulfate, addition of charcoal, clarification, addition of N,N-diethyl-1,1,2,3,3,3-hexafluoro-1-propanamine (Ishikawa Reagent) (about 4.7 g, 0.0211 moles), heating to a temperature of from about 95° C. to about 105° C. for about 6 hours then cooling to a temperature of from about 20° C. to about 25° C., the reaction mixture can produce a solution of 3-(dichloroacetyl)-4(S)-(fluoromethyl)-5(R)-[4-(methylsulfonyl)phenyl]-2-oxazolidinone (Compound Xd). Following addition to sodium hydroxide (about 1.5 g) in water (about 330 mL), separation of the methylene chloride layer, evaporation of the methylene chloride, washing with water and isopropanol then drying, the solution of Compound Xd can yield 3-(dichloroacetyl)-4(S)-(fluoromethyl)-5(R)-[4-(methylsulfonyl)phenyl]-2-oxazolidinone (Compound Xd).

### Example 12

Preparation of Florfenicol (Compound I). 3-(Dichloroacetyl)-4(S)-(fluoromethyl)-5(R)-[4-(methylsulfonyl)phenyl]-2-oxazolidinone (Compound Xd) (about 5 g, 0.130 moles) can be hydrolyzed in tetrahydrofuran (about 50 mL) and water (about 5 mL) containing LiOH at a temperature of from about 25° C. to about 35° C. for about 6 hours to provide a reaction mixture. Following concentration of the solvent, addition of water, filtration of the resulting solid, washing with water and toluene, the reaction mixture can yield Florfenicol (Compound I).

### Example 13

Preparation of Florfenicol (Compound I). 3-(Dichloroacetyl)-4(R)-(hydroxymethyl)-5(R)-[4-(methylsulfonyl)phenyl]-2-oxazolidinone (Compound Vd) (about 5 g, 0.0130 moles) in methylene chloride (about 75 ml) can be reacted with N,N-diethyl-1,1,2,3,3,3-hexafluoro-1-propanamine (Ishikawa Reagent) (about 8.4 g, 0.0197 moles) at a temperature of from about 95° C. to about 105° C. over about 6 hours to provide a reaction mixture. Following cooling to a temperature of from about 20° C. to about 25° C., quenching with about 25% aqueous sodium hydroxide and water (about 75 mL) and separation of the methylene chloride layer, the reaction mixture gives a solution of 3-(dichloroacetyl)-4(S)-(fluoromethyl)-5(R)-[4-(methylsulfonyl)phenyl]-2-oxazolidinone (Compound Xd). Following addition of water (about 25 mL) and p-toluene sulfonic acid with heating to a temperature of from about 30° C. to about 40° C. for about 18 hours, addition of more water (about 50 mL), filtration of the resulting solid, washing with water and toluene then drying, the solution of Compound Xd can yield Florfenicol (Compound I).

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

The words “comprise”, “comprises”, and “comprising” are to be interpreted inclusively rather than exclusively. This interpretation is intended to be the same as the interpretation that these words are given under United States patent law.

The singular forms “a”, “an”, and “the” include plural references, unless the context clearly dictates otherwise. It is intended that each of the patents, patent applications, technical articles and reports, government, trade and industry publications, printed publications, including books and any of the aforementioned publications, mentioned in this patent document be hereby incorporated by reference in its entirety.

1. A process for preparing an oxazolidine-protected aminodiol compound of Formula VI or a pharmaceutically acceptable salt thereof, wherein:
the compound of Formula VI corresponds in structure to: 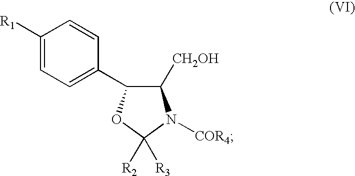
the process comprises:
a) reacting a compound of Formula VII with an oxazolidine-forming solvent to form a reaction mixture, and
b) adding an oxazolidine-forming reagent and oxazolidine-promoting compound to the reaction mixture to form the oxazolidine protected aminodiol of Formula VI;
the compound of Formula VII corresponds in structure to: 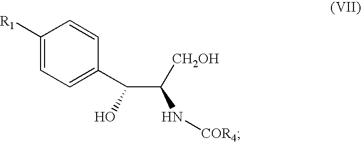
R₁ is hydrogen, methylthio, methylsulfoxy, methylsulfonyl, fluoromethylthio, fluoromethylsulfoxy, fluoromethylsulfonyl, nitro, fluoro, bromo, chloro, acetyl, benzyl, phenyl, halo-substituted phenyl, C₁₋₆ alkyl, C₁₋₆ haloalkyl, C₃₋₈ cycloalkyl, C₂₋₆ alkenyl, C₂₋₆ alkynyl, alkoxy, C₁₋₆ aralkyl, C₂₋₆ aralkenyl, or a C₃₋₇ heterocyclic group;
R₂ is hydrogen, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, aryl, or a C₃₋₇ heterocyclic group;
R₃ is hydrogen, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, aryl, or a C₃₋₇ heterocyclic group; and
R₄ is hydrogen, C₁₋₆ alkyl, C₁₋₆ haloalkyl, C₁₋₆ dihaloalkyl, C₁₋₆ trihaloalkyl, CH₂Cl, CHCl₂, CCl₃, CH₂Br, CHBr₂, CBr₃, CH₂F, CHF₂, CF₃, C₃₋₈ cycloalkyl, C₃₋₈ cyclohaloalkyl, C₃₋₈ cyclodihaloalkyl, C₃₋₆ cyclotrihaloalkyl, C₂₋₆ alkenyl, C₂₋₆ alkynyl, C₁ alkoxy, C₁₋₆ aralkyl, C₂₋₆ aralkenyl, a C₃₋₇ heterocyclic group, benzyl, phenyl, or phenyl alkyl, wherein:
the phenyl or phenyl alkyl can be substituted by one or two halogens, C₁₋₆ alkyl, or C₁₋₆ alkoxy.

2. The process of claim 1, wherein R₂ and R₃ are each methyl.

3. The process of claim 1, wherein the oxazolidine-forming solvent comprises toluene.

4. The process of claim 1, wherein the oxazolidine-forming reagent comprises acetone.

5. The process of claim 4, wherein the oxazolidine-forming solvent comprises toluene and wherein toluene and acetone are present in a toluene-to-acetone ratio of from about 0.5:1 to about 3:1.

6. The process of claim 1, wherein the compound of Formula VI corresponds in structure to Formula III:

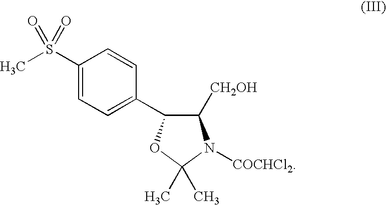

7. The process of claim 1, wherein:
the process further comprises:
fluorinating the compound of Formula VI (or its salt) with a fluorinating agent in the presence of an organic solvent to obtain a compound of Formula VIII, and
hydrolyzing the compound of Formula VIII with an acid or basic catalyst and a solvent to form a compound of Formula IX;
the compound of Formula VIII corresponds in structure to: 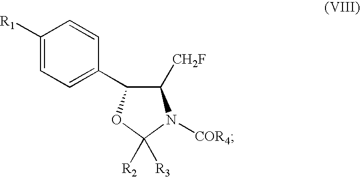
and
the compound of Formula IX corresponds in structure to:

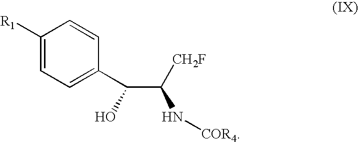

8. The process of claim 7, wherein one or both of the Formula VI compound of the fluorination and the Formula VIII compound of the hydrolyzation are generated in situ.

9. The process of claim 7, wherein the hydrolyzation is performed at a temperature of less than about 80° C.

10. The process of claim 7, wherein the compound of Formula VIII corresponds in structure to Formula VIIId:

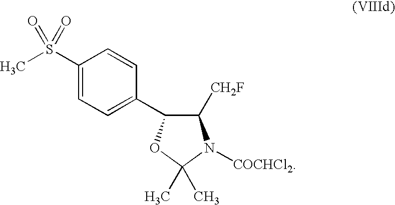

11. The process of claim 7, wherein the acid or basic catalyst comprises p-toluene sulfonic acid.

12. The process of claim 7, wherein the acid or basic catalyst comprises K₂CO₃.

13. A process for preparing an oxazolidinone protected aminodiol compound of Formula V or a pharmaceutically-acceptable salt thereof, wherein:
the compound of Formula V corresponds in structure to: 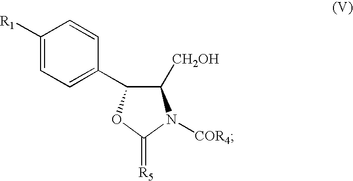
the process comprises:
a) reacting a compound of Formula VII with an oxazolidinone-forming solvent, and
b) adding an oxazolidinone-forming reagent and oxazolidinone-promoting compound to form the oxazolidinone-protected aminodiol of Formula V;
the compound of Formula VII corresponds in structure to: 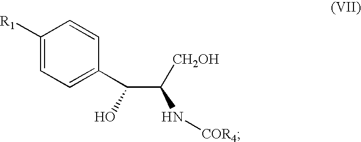
R₁ is hydrogen, methylthio, methylsulfoxy, methylsulfonyl, fluoromethylthio, fluoromethylsulfoxy, fluoromethylsulfonyl, nitro, fluoro, bromo, chloro, acetyl, benzyl, phenyl, halo substituted phenyl, C₁₋₆ alkyl, C₁₋₆ haloalkyl, cycloalkyl, C₂₋₆ alkenyl, C₂₋₆ alkynyl, C₁₋₆ alkoxy, C₁₋₆ aralkyl, C₂₋₆ aralkenyl, or a C₃₋₇ heterocyclic group;
R₄ is hydrogen, C₁₋₆ alkyl, C₁₋₆ haloalkyl, C₁₋₆ dihaloalkyl, C₁₋₆ trihaloalkyl, CH₂Cl, CHCl₂, CCl₃, CH₂Br, CHBr₂, CBr₃, CH₂F, CHF₂, CF₃, C₃₋₈ cycloalkyl, C₃₋₈ cyclohaloalkyl, C₃₋₈ cyclodihaloalkyl, C₃₋₆ cyclotrihaloalkyl, C₂₋₆ alkenyl, C₂₋₆ alkynyl, C₁₋₆ alkoxy, C₁₋₆ aralkyl, C₂₋₆ aralkenyl, a C₃₋₇ heterocyclic group, benzyl, phenyl, or phenyl alkyl, wherein:
the phenyl or phenyl alkyl can be substituted by one or two halogen, C₁₋₆ alkyl, or C₁₋₆ alkoxy; and
R₅ is oxygen, sulfur, or monosubstituted amino.

14. The process of claim 13, wherein the oxazolidinone-forming solvent comprises methanol.

15. The process of claim 13, wherein the oxazolidinone-forming reagent comprises one or both of dimethyl carbonate and diethyl carbonate.

16. The process of claim 13, wherein the compound of Formula V corresponds in structure to Formula Vd:

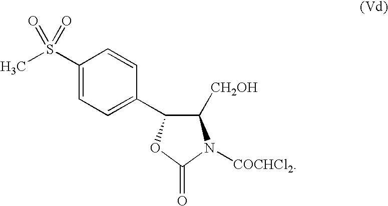

17. The process of claim 13, wherein:
the process further comprises:
fluorinating the compound of Formula V (or its salt) with a fluorinating agent in the presence of an organic solvent to obtain a compound of Formula X, and
hydrolyzing the compound of Formula X with an acid or basic catalyst in a solvent to form the compound of Formula IX;
the compound of Formula X corresponds in structure to: 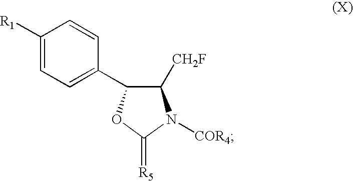
and
the compound of Formula IX corresponds in structure to:

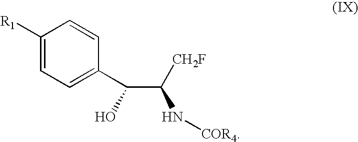

18. The process of claim 17, wherein one or both of the Formula V compound of the fluorination and the Formula X compound of the hydrolyzation are generated in situ.

19. The process of claim 17, wherein the compound of Formula X corresponds in structure to Formula Xd:

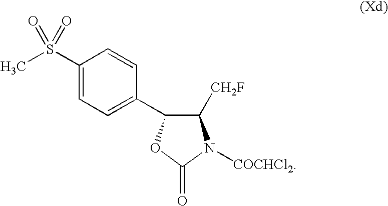

20. The process of claim 17, wherein the acid or basic catalyst comprises methanesulfonic acid.

21. The process of claim 17, wherein the acid or basic catalyst comprises LiOH.

22. The process of claim 17, wherein the hydrolyzation further comprises heating the compound of Formula X with the acid or basic catalyst in a mixture of an organic solvent and water at a temperature of less than about 100° C.

23. The process of claim 1, wherein the compound of Formula VII is thiamphenicol of Formula IV:

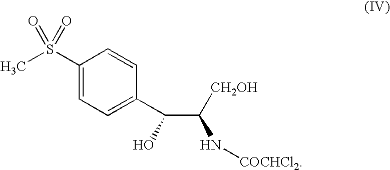

24. The process of claim 1, wherein R₁ is methylsulfonyl.

25. The process of claim 1, wherein R₄ is CHCl₂.

26. The process of claim 1, wherein the oxazolidinone-promoting compound comprises one or both of potassium carbonate and triethylamine.

27. The process of claim 7, wherein the organic solvent for the fluorination comprises methylene chloride.

28. The process of claim 7, wherein the solvent for the hydrolyzation comprises a mixture of an organic solvent and water.

29. The process of claim 7, wherein the solvent for the hydrolyzation comprises one or both of tetrahydrofuran and methylene chloride.

30. The process of claim 7, wherein the process further comprises purifying the compound of Formula IX to obtain purified compound of Formula IX.

31. The process of claim 30, wherein the purification comprises using a mixture comprising water and isopropanol.

32. The process of claim 30, wherein the purification comprises using a cooling temperature of from about 10° C. to about 30° C. to obtain crystallized compound of Formula IX.

33. The process of claim 7, wherein the fluorinating agent comprises N,N-diethyl-1,1,2,3,3,3-hexafluoro-1-propanamine.

34. The process of claim 7, wherein the compound of Formula IX is Florfenicol.

35. A compound of Formula V or a pharmaceutically-acceptable salt thereof, wherein:
the compound of Formula V corresponds in structure to: 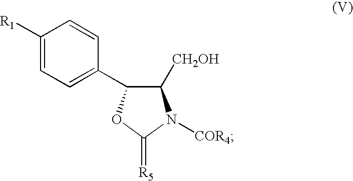
R₁ is methylthio, methylsulfoxy, methylsulfonyl, fluoromethylthio, fluoromethylsulfoxy, fluoromethylsulfonyl, nitro, fluoro, bromo, chloro, acetyl, benzyl, phenyl, halo substituted phenyl, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, or a C₃₋₇ heterocyclic group;
R₄ is hydrogen, C₁₋₆ alkyl, C₁₋₆ haloalkyl, C₁₋₆ dihaloalkyl, C₁₋₆ trihaloalkyl, CH₂Cl, CHCl₂, CCl₃, CH₂Br, CHBr₂, CBr₃CH₂F, CHF₂, CF₃, C₃₋₈ cycloalkyl, C₃₋₈ cyclohaloalkyl, C₃₋₆ cyclodihaloalkyl, C₃₋₆ cyclotrihaloalkyl, C₂₋₆ alkenyl, C₂₋₆ alkynyl, C₁₋₆ alkoxy, C₁₋₆ aralkyl, C₂₋₆ aralkenyl, a C₃₋₇ heterocyclic group, benzyl, phenyl, or phenyl alkyl, wherein:
the phenyl or phenyl alkyl can be substituted by one or two halogens, C₁₋₆ alkyl, or C₁₋₆ alkoxy; and
R₅ is oxygen, sulfur, or monosubstituted amino.

36. The compound or salt of claim 35, wherein the compound of Formula V corresponds in structure to Formula Vd:

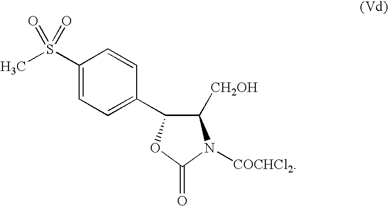

37. A compound of Formula X or a pharmaceutically-acceptable salt thereof, wherein:
the compound of Formula X corresponds in structure to: 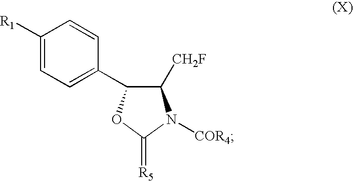
R₁ is hydrogen, methylthio, methylsulfoxy, methylsulfonyl, fluoromethylthio, fluoromethylsulfoxy, fluoromethylsulfonyl, nitro, fluoro, bromo, chloro, acetyl, benzyl, phenyl, halo substituted phenyl, C₁₋₆ alkyl, C₁₋₆ haloalkyl, C₃₋₈ cycloalkyl, C₂₋₆ alkenyl, C₂₋₆ alkynyl, C₁₋₆ alkoxy, C₁₋₆ aralkyl, C₂₋₆ aralkenyl, or a C₃₋₇ heterocyclic group;
R₄ is hydrogen, C₁₋₆ alkyl, C₁₋₆ haloalkyl, C₁₋₆ dihaloalkyl, C₁₋₆ trihaloalkyl, CH₂Cl, CHCl₂, CCl₃, CH₂Br, CHBr₂, CBr₃, CH₂F, CHF₂, CF₃, C₃₋₈ cycloalkyl, C₃₋₈ cyclohaloalkyl, C₃₋₈ cyclodihaloalkyl, C₃₋₈ cyclotrihaloalkyl, C₂₋₆ alkenyl, C₂₋₆ alkynyl, C₁₋₆ alkoxy, C₁₋₆ aralkyl, C₂₋₆ aralkenyl, a C₃₋₇ heterocyclic group, benzyl, phenyl, or phenyl alkyl, wherein:
the phenyl or phenyl alkyl can be substituted by one or two halogens, C₁₋₆ alkyl, or C₁₋₆ alkoxy; and
R₅ is oxygen, sulfur, or monosubstituted amino,
with the proviso that if R₄ is O-t-butyl and R₅ is O, then R₁ is not Br, CH₃SO₂, or CH₃S.

38. The compound or salt of claim 37, wherein the compound of Formula X corresponds in structure to Formula Xd: [chemical structure]
